(19) United States
(12) Patent Application Publication
(10) Pub. No.: US 2022/0334059 A1
(43) Pub. Date: Oct. 20, 2022

(54) MERCURY MEASUREMENT

(71) Applicant: Hach Company, Loveland, CO (US)

(72) Inventor: Amit Das, Fort Collins, CO (US)

(21) Appl. No.: 17/233,942

(22) Filed: Apr. 19, 2021

(51) Int. Cl. G01N 21/64

(52) U.S. Cl. CPC G01N 21/643; G01N 2021/6439; C07D 213/75

(57) ABSTRACT

An embodiment provides a method for measuring mercury in a solution, including: preparing a thiocarbamate-based indicator; introducing the thiocarbamate-based indicator to a solution, wherein the solution contains an amount of mercury and the introducing causes a change in fluorescence of the solution; and measuring the amount of mercury in the solution by measuring a change in intensity of the fluorescence. Other aspects are described and claimed.

### BACKGROUND

This application relates generally to measuring mercury in aqueous or liquid samples, and, more particularly, to the measurement of mercury using a thiocarbamate-based indicator.

Ensuring water quality is critical in a number of industries such as pharmaceuticals and other manufacturing fields. Additionally, ensuring water quality is critical to the health and well-being of humans, animals, and plants which are reliant on the water for survival. One element that is typically measured is mercury. Too much free mercury in water can be harmful to humans or animals, and regulated to be maintained below a regulated or desired level. Therefore, detecting the presence and concentration of mercury in water or other liquid solutions is vital.

### BRIEF SUMMARY

In summary, one embodiment provides a method for measuring mercury in a solution, comprising: preparing a thiocarbamate-based indicator; introducing the thiocarbamate-based indicator to a solution, wherein the solution contains an amount of mercury and the introducing causes a change in fluorescence of the solution; and measuring the amount of mercury in the solution by measuring a change in intensity of the fluorescence.

Another embodiment provides a device which measures mercury in a solution, comprising: a processor; and a memory storing instructions executable by the processor to: prepare a thiocarbamate-based indicator; introduce the thiocarbamate-based indicator to a solution, wherein the solution contains an amount of mercury and the introducing causes a change in fluorescence of the solution; and measure the amount of mercury in the solution by measuring a change in intensity of the fluorescence.

A further embodiment provides a method for measuring mercury in a solution, comprising: preparing a thiocarbamate-based indicator, wherein the thiocarbamate-based indicator comprises a umbelliferone derivative comprising a coumarin based fluorophore; introducing the thiocarbamate-based indicator to a water sample, wherein the solution contains an amount of mercury and the introducing causes a change in fluorescence of the solution; and measuring the amount of mercury in the solution by measuring a change in intensity of the fluorescence, wherein the fluorescence intensity is correlated to a concentration of the mercury in the solution.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates a flow diagram of an example mercury measuring system.

FIG. 2 illustrates a chemical equation of an example thiocarbamate-based indicator for detection of mercury.

FIG. 3 illustrates an example emission spectral change for example mercury concentrations using a thiocarbamate-based indicator.

FIG. 4 illustrates an example fluorescence intensity measurement using a thiocarbamate-based indicator.

FIG. 5 illustrates an example of computer circuitry.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

Conventional methods of mercury measurement in water may have some limitations. For example, mercury measurement may be used to determine the quality of water. High concentrations of mercury may be harmful to animals, humans, and/or plants. Accordingly, as another example, a user or entity may want the mercury in a body of water to be under a particular threshold, therefore, the user may measure the mercury in order to determine if the amount of mercury is under that threshold.

Current methods, systems and kits for mercury measurement may be cumbersome and require many steps. Multiple steps may introduce errors into a mercury measurement. Additionally, mercury measurement may require a preconcentration step. Conventional methods may not be accurate or detect or measure mercury in very low ranges. What is needed is needed is a method to measure mercury without multiple steps and/or preconcentration of a sample.

Accordingly, an embodiment provides a system and method for measuring mercury at ultralow range (ULR) concentrations with improved reagent stability under ambient conditions. The amount of mercury in the water may be less than 200 parts per billion (ppb), 50 ppb, 20 ppb, 5 ppb, 2 ppb, or 1 ppb. Thus, in an embodiment, the method may detect mercury in concentrations below 200 (ppb, and may yield accurate concentration measurement of mercury as low or lower than 50 ppb, 20 ppb, 10 ppb, 5 ppb, 2 ppb, or 1 ppb. In an embodiment, the method may use a fluorometric method. The indicator to give a fluorescence signal may be a thiocarbamate derivative. The thiocarbamate derivative may be a coumarin based fluorophore. The thiocarbamate-based indicator may be a umbelliferone or 4-methylumbelliferone thiocarbamate. In an embodiment, the fluorescence intensity may be correlated to measurement and detection of mercury. In an embodiment, the pH of a solution may be adjusted to activate the reporter or indicator molecule.

The illustrated example embodiments will be best understood by reference to the figures. The following description is intended only by way of example, and simply illustrates certain example embodiments.

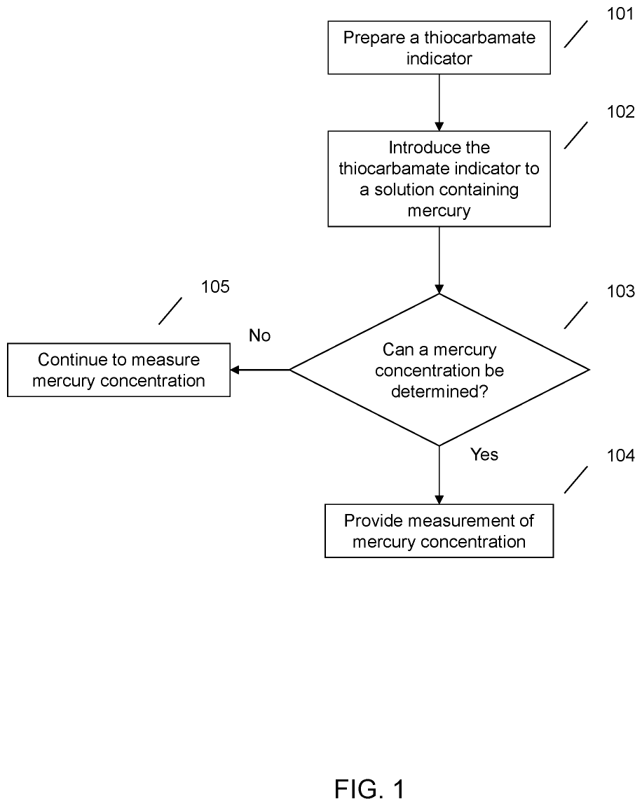

Referring to FIG. 1, an example system and method for detection of mercury in solution is illustrated. In an embodiment, a thiocarbamate-based indicator may be prepared. The thiocarbamate-based indicator may be introduced to a solution containing mercury. In an embodiment, the thiocarbamate-based indicator in the presence of mercury may cause a change in fluorescence intensity of the thiocarbamate-based indicator. The change of fluorescence intensity may be correlated to a concentration of mercury in the solution.

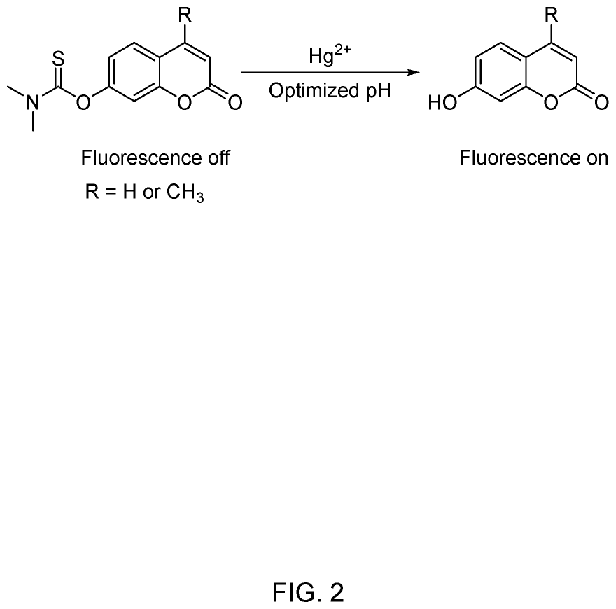

At 101, in an embodiment, a thiocarbamate-based indicator may be prepared. The thiocarbamate may be a thiocarbamate derivative of a coumarin based fluorophore. In an embodiment, the thiocarbamate-based indicator may be umbelliferone or 4-methylumbelliferone thiocarbamate. Referring to FIG. 2, an example reaction of the thiocarbamate-based indicator is illustrated. In an embodiment, the thiocarbamate-based indicator may detect mercury in the ultralow range of less than 200 ppb. The thiocarbamate-based indicator may remain stable for long periods of storage. For example, storage of 4 months may not diminish the accurate determination of mercury in solution.

At 102, in an embodiment, the thiocarbamate-based indicator may be introduced into a solution. The solution may contain mercury or an amount of mercury. The solution may be an aqueous sample which may include a sample from a natural body of water, a holding tank, a processing tank, a pipe, or the like. The solution may be in a continuous flow, a standing volume of liquid, or any combination thereof. In one embodiment, the solution may be introduced to the thiocarbamate-based indicator, for example, a test chamber of the measurement device. In an embodiment, the measurement device may be a hand held device. A hand held device may have advantages such as lower cost, portability, field use, or the like. Alternatively, the measurement device may be a larger bench top device. Introduction of the solution into the measurement device may include placing or introducing the solution into a test chamber manually by a user or using a mechanical means, for example, gravity flow, a pump, pressure, fluid flow, or the like. For example, a water sample for mercury testing may be introduced to a measurement or test chamber using a pump. In an embodiment, valves or the like may control the influx and efflux of the solution into or out of the one or more chambers, if present.

Additionally or alternatively, the measurement device may be present or introduced in a volume of the solution. The measurement device is then exposed to the volume of solution where it may perform measurements. The system may be a flow-through system in which a solution and/or reagents are automatically mixed and measured. Once the sample is in contact with the measurement system, the system may measure the mercury or an amount of mercury of the sample, as discussed in further detail herein. In an embodiment, the measurement device may include one or more chambers in which the one or more method steps may be performed.

In an embodiment, measurement of mercury in water or an aqueous sample may use a thiocarbamate-based indicator. The thiocarbamate-based indicator may comprise umbelliferone and/or 4-methylumbelliferone. Mercury in +2 oxidation state has strong affinity for sulfur and bind efficiently with sulfur containing compounds. In an embodiment, Hg²⁺ may rip the sulfur off from the molecule and produce mercuric sulfide (HgS).

In an embodiment, 7-hydroxy coumarin and its substituted derivatives are highly fluorescent. In an embodiment, when the hydroxy group is being protected using N,N-dimethylthiocarbamoyl group, the molecule becomes very weak fluorescent to nonfluorescent. In an embodiment, upon reaction of the indicator with Hg²⁺ in water, results in the formation of the reporter molecule with increase in fluorescence intensity. (See FIG. 2).

In an embodiment, the pH of the solution may be maintained at a pH of between 5 and 7. In an embodiment, the pH of the solution may be maintained at a pH of approximately 6.0. For example, the pH may be adjusted or titrated to around a pH of 6.0. The concentration of thiocarbamate in the thiocarbamate-based indicator may be between 1 and 10 μM, preferably between 4-6 μM. In an embodiment, a buffer may be added. In an embodiment a phosphate buffer may be added. In an embodiment, increasing the amount of buffer may decrease fluorescence intensity. Saline may be added to the solution. The saline may not affect the fluorescence intensity.

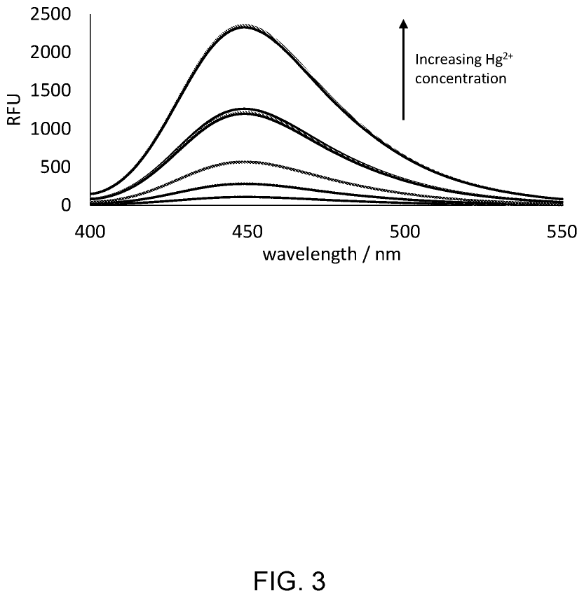

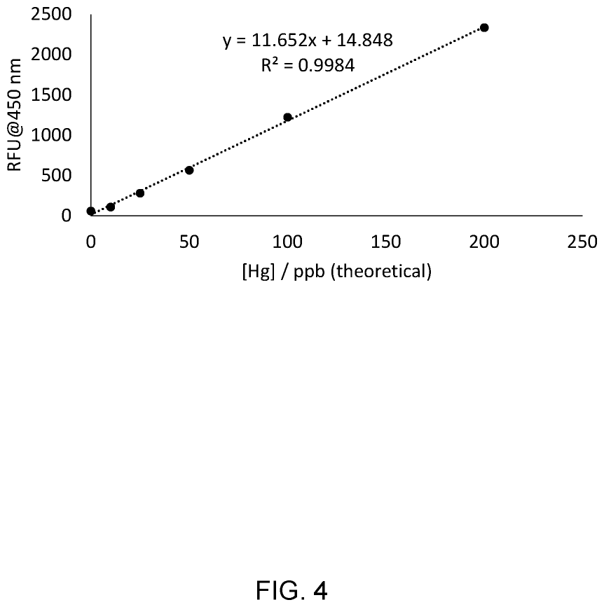

At 103, in an embodiment, the system and method may determine if a mercury concentration or an amount of mercury may be measured. In an embodiment, the presence of mercury in an aqueous solution may cause an increase in fluorescence intensity of the thiocarbamate-based indicator. In an embodiment, the thiocarbamate derivative may be selective for mercury and react with mercury in an aqueous environment releasing a fluorescence active molecule. Examples of this increase in fluorescence intensity and dose response curves for a thiocarbamate-based indicator may be illustrated in FIG. 3 and FIG. 4. Therefore, the fluorescence intensity, of a solution containing mercury may be correlated to the concentration of the mercury in the aqueous solution. Fluorescence curves may be generated for a range of mercury concentrations, for different thiocarbamate-based indicators, for any different condition that may affect absorption or fluorescence values (e.g., temperature, sample content, turbidity, viscosity, measurement apparatus, aqueous sample chamber, etc.), or the like.

In an embodiment, an approximate range of detection of mercury is between 0-200 ppb. Referring to FIG. 3, an emission spectral change by varying mercury concentration or amount in water is illustrated. The reaction mixture was excited at 325 nm wavelength and emission was monitored from 400 nm to 550 nm. FIG. 3 shows the emission spectral change when concentration of Hg²⁺ was varied from 0 ppb to 200 ppb. An increase in fluorescence intensity was observed on increasing concentration of Hg²⁺ in water.

Referring to FIG. 4, in an embodiment a calibration plot for mercury detection in water is illustrated. A dose response curve or calibration plot may be generated by plotting emission intensity at 450 nm wavelength (λex=325 nm) versus the theoretical concentration of Hg²⁺. A linear response was observed for 0-200 ppb concentration range of mercury.

Alternatively or additionally, mercury concentration measurement may be at periodic intervals set by the user or preprogrammed frequencies in the device. Measurement of mercury by a device allows for real time data with very little human involvement in the measurement process. Cleaning of the fluorometric chamber may be required at an unspecified time interval. A programmed calibration curve may be entered into the device.

A chamber, vessel, cell, chamber, or the like may contain an aqueous sample, at least one thiocarbamate-based indicator, and associated reagents such as buffers and/or additives. A device may contain one or more bottles of reagents which contain necessary reagents. The reagents contained in the one or more bottles may be pump fed or gravity fed. The flow of the reagents may be metered to ensure proper volume delivery to the measurement cell. The aqueous sample may be fed through a pressured inlet, a vessel, or the like. The aqueous sample may be introduced into the measurement chamber by a pump or gravity fed. The sampling device may be in series or parallel to an aqueous flow. The device may have a system to ensure proper mixing of the aqueous sample, thiocarbamate-based indicator, and related reagents.

The fluorescence intensity or mercury concentration may be an output upon a device in the form of a display, printing, storage, audio, haptic feedback, or the like. Alternatively or additionally, the output may be sent to another device through wired, wireless, fiber optic, Bluetooth®, near field communication, or the like. An embodiment may use an alarm to warn of a measurement or concentration outside acceptable levels. An embodiment may use a system to shut down water output or shunt water from sources with unacceptable levels of an analyte. For example, an analyte measuring device may use a relay coupled to an electrically actuated valve, or the like.

At 105, in an embodiment, if a concentration of mercury cannot be determined, the system may continue to measure mercury. Additionally or alternatively, the system may output an alarm, log an event, or the like.

At 104, if a concentration or amount of mercury can be determined, the system may provide a measurement of mercury concentration. The system may connect to a communication network. The system may alert a user or a network. This alert may occur whether a mercury measurement is determined or not. An alert may be in a form of audio, visual, data, storing the data to a memory device, sending the output through a connected or wireless system, printing the output or the like. The system may log information such as the measurement location, a corrective action, geographical location, time, date, number of measurement cycles, or the like. The alert or log may be automated, meaning the system may automatically output whether a correction was required or not. The system may also have associated alarms, limits, or predetermined thresholds. For example, if a mercury concentration reaches a threshold. Alarms or logs may be analyzed in real-time, stored for later use, or any combination thereof.

The various embodiments described herein thus represent a technical improvement to conventional mercury measurement techniques. Using the techniques as described herein, an embodiment may use a thiocarbamate-based indicator to measure mercury in solution. Such techniques provide a faster and more accurate method for measuring mercury in an aqueous or water sample.

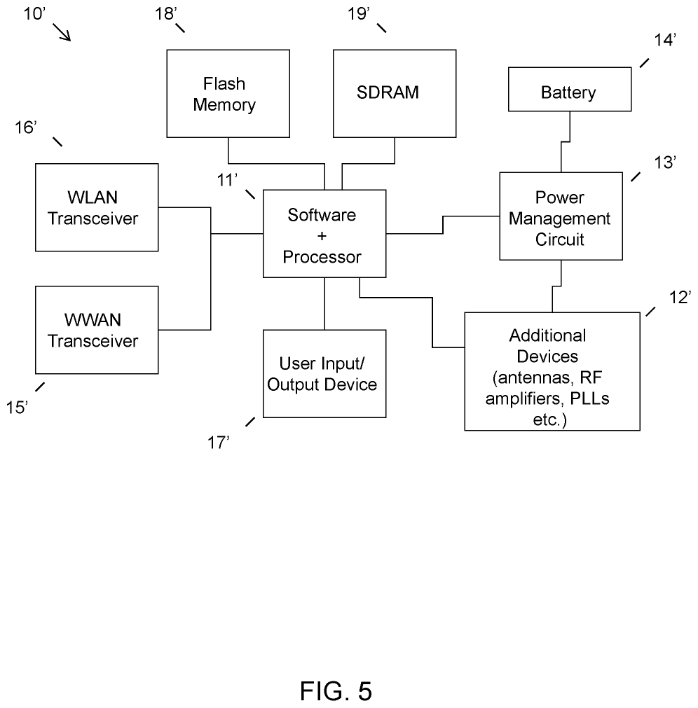

While various other circuits, circuitry or components may be utilized in information handling devices, with regard to an instrument for measurement of mercury according to any one of the various embodiments described herein, an example is illustrated in FIG. 5. Device circuitry 10′ may include a measurement system on a chip design found, for example, a particular computing platform (e.g., mobile computing, desktop computing, etc.) Software and processor(s) are combined in a single chip 11′. Processors comprise internal arithmetic units, registers, cache memory, busses, I/O ports, etc., as is well known in the art. Internal busses and the like depend on different vendors, but essentially all the peripheral devices (12′) may attach to a single chip 11′. The circuitry 10′ combines the processor, memory control, and I/O controller hub all into a single chip 11′. Also, systems 10′ of this type do not typically use SATA or PCI or LPC. Common interfaces, for example, include SDIO and I2C.

There are power management chip(s) 13′, e.g., a battery management unit, BMU, which manage power as supplied, for example, via a rechargeable battery 14′, which may be recharged by a connection to a power source (not shown). In at least one design, a single chip, such as 11′, is used to supply BIOS like functionality and DRAM memory.

System 10′ typically includes one or more of a WWAN transceiver 15′ and a WLAN transceiver 16′ for connecting to various networks, such as telecommunications networks and wireless Internet devices, e.g., access points. Additionally, devices 12′ are commonly included, e.g., a transmit and receive antenna, oscillators, PLLs, etc. System 10′ includes input/output devices 17′ for data input and display/rendering (e.g., a computing location located away from the single beam system that is easily accessible by a user). System 10′ also typically includes various memory devices, for example flash memory 18′ and SDRAM 19′.

It can be appreciated from the foregoing that electronic components of one or more systems or devices may include, but are not limited to, at least one processing unit, a memory, and a communication bus or communication means that couples various components including the memory to the processing unit(s). A system or device may include or have access to a variety of device readable media. System memory may include device readable storage media in the form of volatile and/or nonvolatile memory such as read only memory (ROM) and/or random access memory (RAM). By way of example, and not limitation, system memory may also include an operating system, application programs, other program modules, and program data. The disclosed system may be used in an embodiment to perform measurement of mercury of an aqueous sample.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device, where the instructions are executed by a processor. In the context of this document, a storage device is not a signal and “non-transitory” includes all media except signal media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and products according to various example embodiments. It will be understood that the actions and functionality may be implemented at least in part by program instructions. These program instructions may be provided to a processor of a device, e.g., a hand held measurement device, or other programmable data processing device to produce a machine, such that the instructions, which execute via a processor of the device, implement the functions/acts specified.

It is noted that the values provided herein are to be construed to include equivalent values as indicated by use of the term “about.” The equivalent values will be evident to those having ordinary skill in the art, but at the least include values obtained by ordinary rounding of the last significant digit.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method for measuring mercury in a solution, comprising:
preparing a thiocarbamate-based indicator;
introducing the thiocarbamate-based indicator to a solution, wherein the solution contains an amount of mercury and the introducing causes a change in fluorescence of the solution; and
measuring the amount of mercury in the solution by measuring a change in intensity of the fluorescence.

2. The method of claim 1, wherein the thiocarbamate-based indicator comprises a umbelliferone derivative.

3. The method of claim 1, wherein the thiocarbamate-based indicator comprises a 4-methylumbelliferone.

4. The method of claim 1, wherein the thiocarbamate-based indicator comprises a coumarin based fluorophore.

5. The method of claim 1, wherein the solution comprises a water sample.

6. The method of claim 1, wherein the amount of mercury removes the sulfur from the thiocarbamate-based indicator increasing fluorescence intensity of the thiocarbamate-based indicator.

7. The method of claim 1, wherein the amount of mercury comprises an amount less than 200 ppb.

8. The method of claim 1, wherein the measuring further comprises titrating a pH of the solution to around a pH of 6.

9. The method of claim 1, wherein the fluorescence intensity is correlated to a concentration of the mercury in the solution.

10. The method of claim 1, wherein the measuring comprises measuring fluorometry with a hand-held device.

11. A device which measures mercury in a solution, comprising:
a processor; and
a memory storing instructions executable by the processor to:
prepare a thiocarbamate-based indicator;
introduce the thiocarbamate-based indicator to a solution, wherein the solution contains an amount of mercury and the introducing causes a change in fluorescence of the solution; and
measure the amount of mercury in the solution by measuring a change in intensity of the fluorescence.

12. The device of claim 11, wherein the thiocarbamate-based indicator comprises a umbelliferone derivative.

13. The device of claim 11, wherein the thiocarbamate-based indicator comprises a 4-methylumbelliferone.

14. The device of claim 11, wherein the thiocarbamate-based indicator comprises a coumarin based fluorophore.

15. The device of claim 11, wherein the solution comprises a water sample.

16. The device of claim 11, wherein the amount of mercury removes the sulfur from the thiocarbamate-based indicator increasing fluorescence intensity of the thiocarbamate-based indicator.

17. The device of claim 11, wherein the amount of mercury comprises an amount less than 200 ppb.

18. The device of claim 11, wherein the measuring further comprises titrating a pH of the solution to around a pH of 6.

19. The device of claim 11, wherein the fluorescence intensity is correlated to a concentration of the mercury in the solution.

20. A method for measuring mercury in a solution, comprising:
preparing a thiocarbamate-based indicator, wherein the thiocarbamate-based indicator comprises a umbelliferone derivative comprising a coumarin based fluorophore;
introducing the thiocarbamate-based indicator to a water sample, wherein the solution contains an amount of mercury and the introducing causes a change in fluorescence of the solution; and
measuring the amount of mercury in the solution by measuring a change in intensity of the fluorescence, wherein the fluorescence intensity is correlated to a concentration of the mercury in the solution.
